(19) United States
(12) Patent Application Publication
(10) Pub. No.: US 2021/0038871 A1
(43) Pub. Date: Feb. 11, 2021

(54) FAST-SWELLING, HIGHLY-SWELLABLE, ROBUST HYDROGEL BALLOONS

(71) Applicant: Massachusetts Institute of Technology, Cambridge, MA (US)

(72) Inventors: Xuanhe Zhao, Allston, MA (US); Hyunwoo Yuk, Cambridge, MA (US); Xinyue Liu, Cambridge, MA (US); Shaoting Lin, Cambridge, MA (US); German Alberto Parada Hermandez, Cambridge, MA (US)

(21) Appl. No.: 16/965,633

(22) PCT Filed: Jan. 15, 2019
(86) PCT No.: PCT/US19/13636

§ 371 Date: Jul. 29, 2020

Related U.S. Application Data
(60) Provisional application No. 62/623,695, filed on Jan. 30, 2018.

(51) Int. Cl. A61M 31/00; A61F 5/00

(52) U.S. Cl. CPC A61M 31/002; A61M 2210/1053; A61F 5/003

(57) ABSTRACT

An expandable hydrogel structure formed of a housing with a superabsorbent material disposed and sealed therein, particularly wherein the housing is fabricated of a hydrogel membrane with a plurality of macropores providing fluid communication between the superabsorbent material and an exterior of the housing. Exposure of the superabsorbent material to an expansion trigger expands the housing from an initial size to an expanded size that is at least about 50 times to at least about 100 times the initial size. One or more therapeutic agents can further be disposed within the housing to provide controlled release of the therapeutic agents from the expanded housing for extended periods.

### CROSS-REFERENCE TO RELATED APPLICATIONS

This application is a national phase entry of PCT/US19/13636, filed Jan. 15, 2019 and entitled FAST-SWELLING, HIGHLY-SWELLABLE, ROBUST HYDROGEL BALLOONS, which claims priority to U.S. Provisional Patent Application Ser. No. 62/623,695, filed on Jan. 30, 2018 and entitled, FAST-SWELLING, HIGHLY-SWELLABLE, ROBUST HYDROGEL BALLOONS. The subject matter of the prior applications are being incorporated in their entirety by reference herein.

### STATEMENT OF FEDERAL SUPPORT

This invention was made with Government support under Grant No. N00014-14-1-0619 awarded by the Office of Naval Research, and Grant No. CMMI-1532136 awarded by the National Science Foundation. The Government has certain rights in the invention.

### FIELD OF THE INVENTION

The present invention relates to an expandable hydrogel, particularly an expandable and anti-fatigue hydrogel structure that swells into a hydrogel balloon-like structure when ingested, implanted, or otherwise inserted into a target site. The expandable hydrogel expands with a high speed and large ratio to form a mechanically robust yet compliant hydrogel balloon-like structure which remains stable for extended periods of time and which can be deflated on demand for removal.

### BACKGROUND OF THE INVENTION

Obesity has become an epidemic in the United States, affecting both adults and children. There is also evidence that obesity is becoming an issue in many other parts of the world. Despite an awareness of this problem and efforts toward addressing it, obesity rates continue to multiply. It is well known that obesity increases the risk of numerous diseases, including, for example, diabetes, cancer, coronary heart disease, hypertension, sleep apnea, stroke, gastroesophageal reflux disease, and gall bladder disease.

Current approaches for reducing obesity include a change in lifestyle, weight loss drugs, and in surgical options (such as bariatric surgery), and weight loss drugs. Lifestyle change alone has been shown to lead generally to limited success. Combining weight loss drugs with lifestyle change has been shown to be more effective, but there are some associated risks. Surgical options, while effective, are costly and carry serious risk.

More recently, endoscopic insertion of intragastric balloons has shown to be promising. In particular, a deflated balloon with an attached inflation catheter is inserted into the stomach using an endoscopic device. Once positioned, the balloon is inflated by flowing a fluid or gas into the balloon through the catheter. This procedure must be performed in a hospital setting, during which the patient is sedated. When in place, the intragastric balloon provides the sensation of feeling fuller faster, which causes a patient to eat less. Such balloons must typically be removed after 6 months, during an in-hospital procedure performed under sedation. While there are current techniques available, there is still a need for improved methods for treating obesity.

Besides the obesity control, the physiological signals inside the gastrointestinal (GI) tract are of importance for biomedical diagnosis and treatment. However, the short stay in GI tract limits the study of the long-term biosignal collection in the digestive system. For example, IntelliCap® and gas sensing capsule can only provide signal information for less than 1 day. It is urgent to develop a device that is capable for the long-term data sampling inside the body.

### SUMMARY OF THE INVENTION

According to one aspect, an embodiment of the present invention provides an expandable hydrogel structure comprising a housing fabricated of a hydrogel membrane, the housing having a cavity therein; a superabsorbent material disposed within the housing cavity, the superabsorbent material having an initial superabsorbent size; and a plurality of macropores in the hydrogel membrane. The housing has an initial housing size, as well as an expanded housing size upon exposure of the superabsorbent material to an expansion trigger. The expanded housing size is at least about 50 times to at least about 100 times the initial housing size.

According to another aspect, an embodiment of the present invention provides a weight-loss balloon for placement in a patient's stomach comprising a housing fabricated of a hydrogel membrane, the housing having a cavity therein; a superabsorbent material disposed and sealed within the housing cavity, the superabsorbent material having an initial superabsorbent size; and a plurality of macropores in the hydrogel membrane providing fluid communication between the superabsorbent material within the cavity and an exterior of the housing. The housing has an initial housing size, and an expanded housing size upon exposure of the superabsorbent material to an expansion trigger, the expanded housing size being at least about 50 times to at least about 100 times the initial housing size.

Embodiments according to these two aspects can include one or more of the following features. The superabsorbent material is sealed within the housing, and the plurality of macropores provides communication between an exterior of the housing and the superabsorbent material. The superabsorbent material has an initial superabsorbent size greater than a size of the plurality of macropores, and the macropores are sized for fast passage of the expansion trigger therethrough. The expansion trigger is absorbable by the superabsorbent material. Absorption of the expansion trigger expands the superabsorbent material from an initial superabsorbent size to an expanded superabsorbent size. The expanded superabsorbent size stretches the hydrogel membrane to the expanded housing size. The hydrogel membrane is fabricated of an anti-fatigue polymer network infiltrated with water, which resists a crack propagation under cyclic loading. The anti-fatigue polymer network is selected from polyvinyl alcohol (PVA), polyacrylamide (PAAm), polyethylene glycol (PEG), polyethylene glycol derivatives, poly-N,N-dimethylacrylamide (DMMA), cellulose, collagen, gelatin, agar, agarose, dextran, alginate, hyaluronan, chitosan, nanocomposites, and combinations thereof. The polyethylene glycol derivatives are selected from acrylated PEG, methacrylated PEG, PEG norbornene, PEG diacrylate, PEG dimethacrylate, and combinations thereof. The antifatigue polymer network includes one or more nanostructures. The superabsorbent material is selected from charged polymer networks. The superabsorbent material is selected from polyacrylic acid (PAA), poly(2-acrylamido-2-methyl-1-propanesulfonic acid) (PAMPS), poly [2-(methacryloyloxy)ethyl] trimethyl ammonium chloride), polyethylenimine, poly(vinylbenzyl)trimethylammonium chloride, poly(3-acrylamidopropyl)trimethylammonium chloride, polydimethylaminoethyl methacrylate, and combinations thereof. The expansion trigger is water and/or gastric fluid. The expandable hydrogel structure is an ingestible pill. The expandable hydrogel structure is an extended drug release device. The expandable hydrogel structure comprises an electronic sensor for recording body signals (e.g., gastrointestinal temperature, gastric juice pH, and glucose level). The expandable hydrogel structure further comprises one or more therapeutic agents disposed within the cavity. The one or more therapeutic agents are disposed within the cavity such that the one or more therapeutic agents are released from the expanded housing. The expanded housing is configured to remain expanded for at least fourteen days. The hydrogel membrane is configured to provide release of the one or more therapeutic agents from the expanded housing by diffusion through the hydrogel membrane and/or by release through the plurality of macropores in the hydrogel membrane. The hydrogel membrane provides controlled release of the one or more therapeutic agents from the expanded housing. The controlled release releases the one or more therapeutic agents from the expanded housing for at least fourteen days. The one or more therapeutic agents is released from the expanded housing on a daily basis for at least fourteen days. The expandable hydrogel structure is an extended therapeutic agent release device, and wherein the hydrogel membrane is configured to provide controlled release of the one or more therapeutic agents from the expanded housing for at least fourteen days. The hydrogel membrane is configured to provide controlled release of the one or more therapeutic agents from the expanded housing on a daily basis for at least fourteen days.

According to another aspect, an embodiment of the present invention provides a method for placing a weight-loss balloon within a patient's stomach comprising (1) administering to a patient an ingestible pill comprising a housing fabricated of a hydrogel membrane, the housing having a cavity therein; a superabsorbent material disposed and sealed within the housing cavity, the superabsorbent material having an initial superabsorbent size; and a plurality of macropores in the hydrogel membrane providing fluid communication between the superabsorbent material within the cavity and an exterior of the housing; (2) allowing gastric juice to pass through the plurality of macropores and allowing the superabsorbent material to absorb one or more expansion triggers in the gastric juice to thereby form a swollen superabsorbent material; and (3) allowing the swollen superabsorbent material to stretch the hydrogel membrane and form a weight-loss balloon structure. The weight-loss balloon structure is at least about 50 times to at least about 100 times larger than the ingestible pill.

According to another aspect, an embodiment of the present invention provides a method for placing an expandable hydrogel structure within a patient's stomach comprising (1) administering to a patient an ingestible pill comprising a housing fabricated of a hydrogel membrane, the housing having a cavity therein, a superabsorbent material disposed and sealed within the housing cavity, the superabsorbent material having an initial superabsorbent size, and a plurality of macropores in the hydrogel membrane providing fluid communication between the superabsorbent material within the cavity and an exterior of the housing; (2) allowing an expansion trigger to pass through the plurality of macropores and allowing the superabsorbent material to absorb the expansion trigger to thereby form a swollen superabsorbent material; and (3) allowing the swollen superabsorbent material to stretch the hydrogel membrane and form an expanded hydrogel balloon structure. The expanded hydrogel balloon structure is at least about 50 times to at least about 100 times larger than the ingestible pill.

Embodiments according to these two aspects can include one or more of the following features. The method further includes removing the weight-loss balloon structure from the patient's stomach by administering a deflation trigger to the patient's stomach, allowing the deflation trigger to pass though the plurality of macropores and react with the swollen superabsorbent material to thereby de-swell the swollen superabsorbent material. The deflation trigger comprises one or more high valent ions. The deflation trigger comprises one or more calcium ions. The ingestible pill further comprises one or more therapeutic agents disposed within the cavity, and the method further comprises after allowing the swollen superabsorbent material to stretch the hydrogel membrane and form the expanded hydrogel balloon structure, allowing release of the one or more therapeutic agents from the expanded housing. The method further comprises controlling the release of the one or more therapeutic agents from the expanded housing. The method further comprises allowing the expanded housing to remain expanded for at least fourteen days, wherein controlling the release of the one or more therapeutic agents from the expanded housing comprises releasing the one or more therapeutic agents from the expanded housing for at least fourteen days. The expanded housing is allowed to remain expanded for at least three weeks. The expanded housing is allowed to remain expanded for at least four weeks. Controlling the release of the one or more therapeutic agents from the expanded housing comprises releasing the one or more therapeutic agents from the expanded housing on a daily basis for at least fourteen days. Allowing release of the one or more therapeutic agents from the expanded housing comprises allowing diffusion of the one or more therapeutic agents through the hydrogel membrane and/or allowing release of the one or more therapeutic agents through the plurality of macropores in the hydrogel membrane.

According to another aspect, an embodiment of the present invention provides a method for providing extended release of one or more therapeutic agents to a target site comprising (1) placing an expandable hydrogel structure within the target site, the expandable hydrogel structure comprising a housing fabricated of a hydrogel membrane, the housing having a cavity therein, a superabsorbent material disposed and sealed within the housing cavity, the superabsorbent material having an initial superabsorbent size, a plurality of macropores in the hydrogel membrane providing fluid communication between the superabsorbent material within the cavity and an exterior of the housing, and one or more therapeutic agents disposed within the cavity; (2) allowing an expansion trigger to pass through the plurality of macropores and allowing the superabsorbent material to absorb the expansion trigger to thereby form a swollen superabsorbent material; (3) allowing the swollen superabsorbent material to stretch the hydrogel membrane and form an expanded hydrogel balloon structure configured to remain expanded for at least fourteen days; and (4) allowing release of the one or more therapeutic agents from the expanded hydrogel balloon structure for at least fourteen days. The expanded hydrogel balloon structure is at least about 50 times to at least about 100 times larger than the expandable hydrogel structure.

Embodiments according to this aspect can include one or more of the following features. The expanded housing is allowed to remain expanded for at least three weeks. The expanded housing is allowed to remain expanded for at least four weeks. The one or more therapeutic agents are released from the expanded hydrogel balloon structure on a daily basis for at least fourteen days. The method further comprises, after allowing release of the one or more therapeutic agents from the expanded hydrogel balloon structure for at least fourteen days, exposing the expanded hydrogel balloon structure to a deflation trigger, allowing the expanded balloon structure to shrink to a shrunken structure, and removing the shrunken structure from the target site. The expandable hydrogel structure is an ingestible or implantable pill. The target site is a stomach of a patient.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

The accompanying drawings are included to provide a further understanding of the embodiments of invention, and are incorporated in and constitute a part of this specification. The components in the drawings are not necessarily to scale, emphasis instead being placed upon clearly illustrating the principles of the embodiments of the present invention. The drawings illustrate embodiments of the invention and, together with the description, serve to explain the principles of the embodiments of the invention.

FIGS. 1a-f schematically illustrate a design of the expandable hydrogel pill as an ingestible device of an embodiment of the present invention.

FIGS. 2a-h illustrate high-speed and high-ratio expansion of the expandable hydrogel devices of an embodiment of the present invention.

FIGS. 3a-b illustrate air-dried and freeze-dried polyacrylamide-agar hydrogels of an embodiment of the present invention.

FIGS. 4a-d illustrate the deswelling of the expanded hydrogel devices of an embodiment of the present invention.

FIGS. 5a-c graphically depict the ex vivo performance of the expandable hydrogel devices of an embodiment of the present invention in a plastic stomach model containing porcine gastric fluid.

FIGS. 6a-d depict the comparison of long-term strength under cyclic tensile test between two hydrogel membranes of an embodiment of the present invention.

FIGS. 7a-g illustrate the mechanical robustness of the expandable hydrogel device of an embodiment of the present invention.

FIGS. 8a-d illustrate the mechanical robustness of the expandable hydrogel device, of an embodiment of the present invention, in water.

FIGS. 9a-f illustrate the cell compatibility of the expandable hydrogel devices of an embodiment of the present invention.

FIG. 10 illustrates the fabrication of the expandable hydrogel devices of an embodiment of the present invention with a cube shape, where the superabsorbent particles and a porous hydrogel membrane are assembled into an expandable hydrogel device with cube shape.

FIGS. 11a-c illustrate in vitro sensing and drug release capacity of the expandable hydrogel devices of an embodiment of the present invention.

### DETAILED DESCRIPTION

The following definitions are useful for interpreting terms applied to features of the embodiments disclosed herein, and are meant only to define elements within the disclosure.

As used herein, the terms “expandable” and “swellable”, when describing the hydrogels of the embodiments of the present invention, all refer to a change in size of the hydrogel structure from an initial size to a size larger than the initial size by at least about 50 times to at least about 100 times. The change in size can be accompanied with a change in overall shape or the shape may remain substantially the same (i.e., no significant visible change to the naked eye in the overall shape) with the increase in size. The terms “expandable” and “swellable” may be used interchangeably herein, with the term “swellable” being used to generally refer to such structures.

As used herein, an “assembled hydrogel structure” refers to the hydrogel membrane which has been formed into a desired housing shape, loaded with a predetermined amount of superabsorbent polymer, sealed, and crumpled or otherwise compressed for use into a desired insertion, ingestion or implantation size and/or shape.

As used herein, the term “pill” or “capsule” refers to an assembled hydrogel structure which has been compressed into a structure and dimension consistent with standard ingestible pills or capsules.

As used herein, the term “balloon” or “balloon-like structure” refers to the expanded or swollen hydrogel structure, which comprises the stretchable hydrogel membrane housing the swollen superabsorbent (the membrane being in a stretched form to accommodate the swollen superabsorbent). This balloon or balloon-like structure has a large overall size compared to the original hydrogel structure prior to expansion/swelling (e.g., at least about 100 times larger).

As used herein, “expansion trigger” refers to any substance that causes the assembled hydrogel structure to swell. In particular, the assembled hydrogel structure contains a superabsorbent composition that absorbs a particular trigger, which results in swelling of the superabsorbent. This expansion trigger, in particular, is water. It is to be noted that in addition to pure water, substances which contain water (e.g., where exposure of the embodiment of the present invention assembled hydrogel structure to that substance results in the water component in the substance coming into contact with the superabsorbent composition to cause swelling of the superabsorbent) may also be considered an “expansion trigger”. As such, for example, if the embodiment of present invention as an assembled hydrogel structure is a pill that is ingested, the pill passes into the stomach. While in the stomach, the pill is surrounded by gastric fluid, which contains water. The water component is absorbed by the superabsorbent contained in the pill, resulting in the desired swelling of the pill into a balloon-like structure.

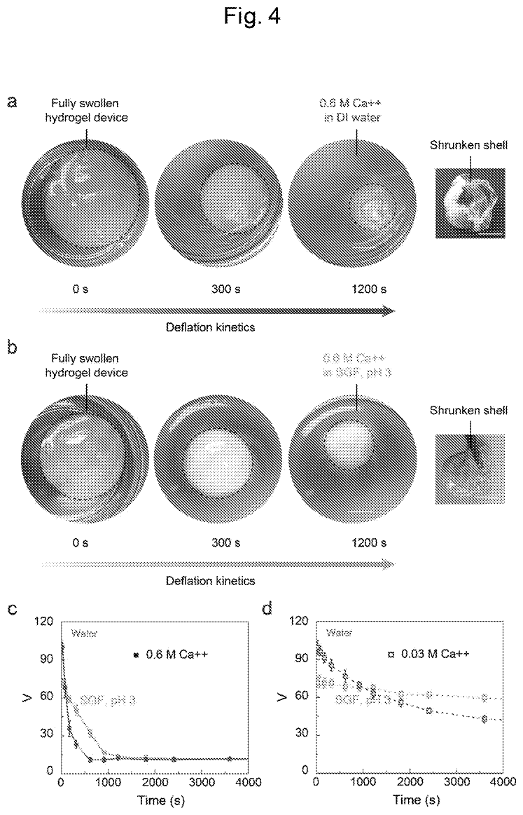

As used herein, the term “deflation” refers to reducing the size of the expanded hydrogel to facilitate removal from a site. The expanded hydrogels may be deflated to the same or substantially the same size as the initial hydrogel structure, but typically need only be deflated to an extent that allows smooth passage through the pylorus for ingestible swellable hydrogel structures. In exemplary embodiments (e.g., FIG. 4), the swollen hydrogel balloon-like structure deflates or decreases in size from the swollen size by around 95% the swollen size.

As used herein, “deflation trigger” refers to any substance that causes the expanded or inflated hydrogel structure to reduce in size for removal from a target site. Such triggers may include high valent ions, such as calcium and iron ions (e.g., Ca′ and Fe′).

As used herein, “crack propagation” is a term known in the art, and refers to a situation where a crack is intentionally made in the hydrogel structure (e.g., by a razor blade or similar tool), and wherein the length of this crack increases when the material is subjected to use or experimental testing (e.g., cyclic loading). Typically, the crack that is made is visible to the naked eye. For example, a general measurement of the crack prior to use or experimental testing is about 0.5-2 mm in length and about 150-250 μm thick. In order to determine whether the length of the crack increases, a high resolution camera is typically used to record and measure the total crack length after use or experimental testing. As such, measurable increase in crack length will depend upon the resolution of the camera. For example, if using a high resolution camera with a spatial resolution of 20 μm, an increase in the crack length would need to be greater than 20 μm to be visible and, thus considered crack propagation.

The present invention generally provides expandable hydrogel structures, which have an initial size and shape and which increase in size upon exposure to an expansion trigger. With this increase in size, the shape may remain substantially the same or it may also change.

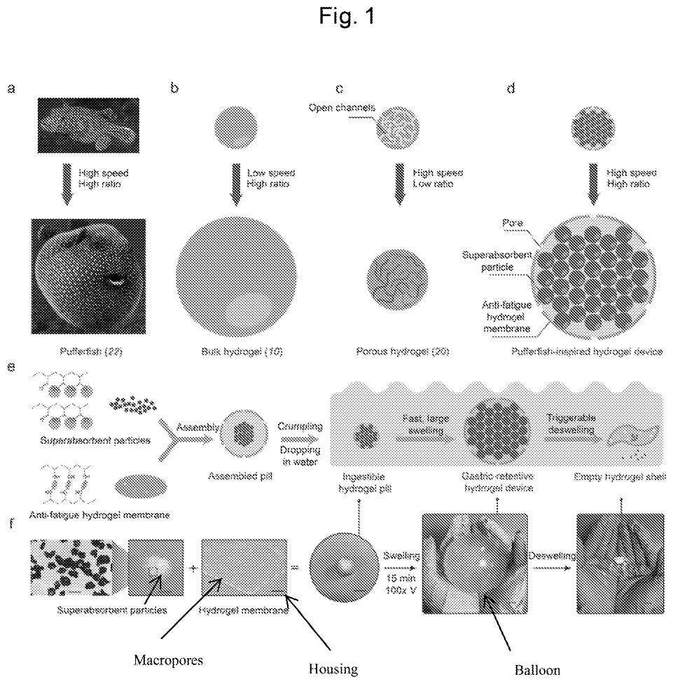

FIGS. 1a-f schematically illustrate a design of the expandable hydrogel pill as an ingestible device, where FIG. 1a illustrates a pufferfish inflates its body into a large ball by rapidly imbibing water, FIG. 1b illustrates bulk hydrogels swell in water with a low swelling speed, FIG. 1c illustrates porous hydrogels swell in water with a low swelling ratio, FIG. 1d illustrates the designed expandable hydrogel swells in water with both a high speed and a high ratio, FIG. 1e schematically illustrates the fabrication process and working principle of the expandable hydrogel device, and FIG. 1f illustrates the fabrication process and working principle of the expandable hydrogel device.

According to an embodiment of the invention, the expandable hydrogel structure is in the form of a hydrogel pill or capsule that is ingestible. Upon ingestion, the hydrogel pill expands into a balloon-like hydrogel structure (e.g., see FIG. 1f). Such expansion occurs at a high speed and ratio. In particular, the hydrogel pills expand to their final size within about 15 minutes, more preferably within about 14 minutes, more preferably within about 13 minutes, more preferably within about 12 minutes, more preferably within about 11 minutes, and even more preferably within about 10 minutes of being exposed to an expansion trigger. The final size may be the maximum size to which the assembled hydrogel structure can possibly expand (i.e. “full size”) or it may be somewhat less than the maximum size (e.g., at least about 80%, at least about 85%, at least about 90%, at least about 95% their maximum size).

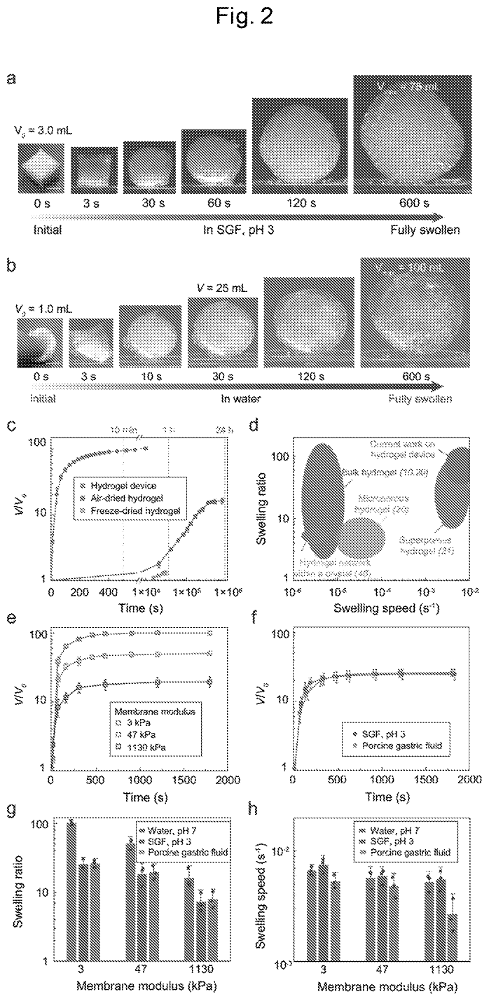

For example, FIGS. 2a-h illustrate embodiments of the high-speed and high-ratio expansion of the expandable hydrogel devices according to the present invention. As shown, FIG. 2a graphically depicts the time-lapse images of the hydrogel swelling in simulated gastric fluid (SGF), FIG. 2b graphically depicts the time-lapse images of the expandable hydrogel device swelling in water (pH 7), FIG. 2c illustrates volume changes of the expandable hydrogel device, air-dried hydrogel, and freeze-dried hydrogel of the same size as a function of swelling time in water, FIG. 2d illustrates the comparison of the swelling ratios and speeds in water between the expandable hydrogel device in current work and previously reported hydrogels, FIG. 2e illustrates volume changes of the expandable hydrogel devices with various membrane moduli as functions of swelling time in water, FIG. 2f illustrates volume changes of the expandable hydrogel devices (membrane modulus 3 kPa) as functions of swelling time in porcine gastric fluid and SGF, FIG. 2g illustrates swelling ratios of the expandable hydrogel devices with various membrane moduli in water, SGF, and porcine gastric fluid, and FIG. 2h illustrates swelling speeds of the expandable hydrogel devices with various membrane moduli in water, SGF, and porcine gastric fluid.

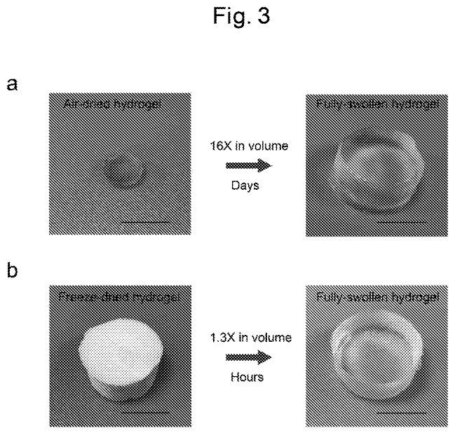

FIGS. 3a-b illustrate air-dried and freeze-dried polyacrylamide-agar hydrogels, where FIG. 3a illustrates the air-dried sample without pores swells in water with high ratio and low speed, and FIG. 3b illustrates the freeze-dried samples with pores swells in water with high speed and low ratio.

According to embodiments of the invention, the assembled hydrogel structures expand to at least about 50 times in volume from the initial size (i.e., prior to any expansion), more preferably at least about 55 times, more preferably at least about 60 times, more preferably at least about 65 times, more preferably at least about 70 times, more preferably at least about 75 times, more preferably at least about 80 times, more preferably at least about 85 times, more preferably at least about 90 times, more preferably at least about 95 times, and even more preferably at least about 100 times volume expansion from the initial size prior to expansion. According to a particularly preferred embodiment, the assembled hydrogel structure is in the form of a pill or capsule that expands at least about 100 times in volume from an initial size upon ingestion within about 10 minutes of exposure to an expansion trigger (FIG. 2).

According to embodiments of the invention, when orally administered, the hydrogel pill or capsule expands into a hydrogel balloon-like structure in the gastric juice of the stomach (and, thus, the water molecules in gastric juice of the stomach act as the expansion trigger). These expanded structures are preferably monolithic and robust, yet compliant. Such expanded hydrogels are capable of maintaining their expanded forms in the stomach (or other target insertion site) for extended periods of time, preferably at least about three weeks, and more preferably for at least about four weeks. In addition, the expanded hydrogel structures can be deflated on demand. In particular, by exposing the expanded hydrogel to a suitable deflation trigger effective in deflating or shrinking the hydrogel structure (e.g., high valent ions), the expanded hydrogel structure shrinks in size so that it can be removed (e.g., exiting through pylorus) (FIG. 4a-c and 5c-b). Such shrinking or deflating may reduce the expanded hydrogel structure back to the same or substantially same size (i.e., no more than about 10% greater than the initial size, more preferably no more than about 8% the initial size, more preferably no more than about 7% the initial size, more preferably no more than about 6% the initial size, and more preferably no more than about 5% the initial size).

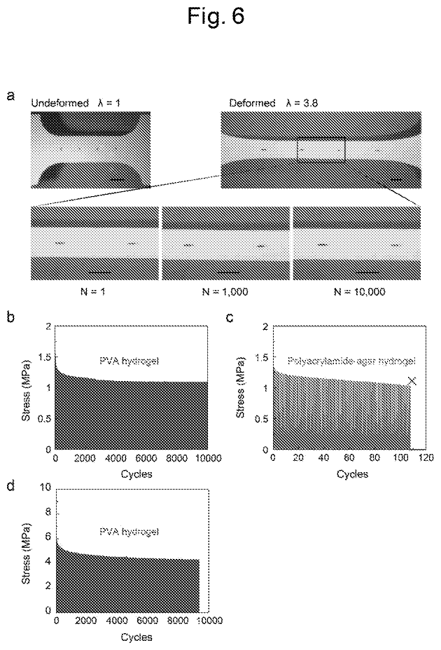

The expandable hydrogel structures of embodiments of the present invention are formed of an anti-fatigue and porous hydrogel membrane which encapsulates a superabsorbent polymer material. In particular, the anti-fatigue and porous hydrogel is formed into a housing with a cavity therein in which the superabsorbent polymer material is disposed. As such, the superabsorbent polymer is sealed within the housing. The hydrogel membrane is fabricated of a polymer network that maintains high elasticity of the hydrogel but can resist crack propagation after prolonged cycles of loads. In particular, the hydrogel membrane is fabricated of a polymer network that is characterized in that when a crack is intentionally made in the hydrogel structure (e.g., by a razor blade or similar tool), the length of this crack does not increases (i.e., no measurable increase in the crack length is detected) when the material us subjected to use or experimental testing (e.g., cyclic loading). In order to determine whether the length of the crack increases, a high resolution camera is typically used to record and measure the total crack length after use or experimental testing. As such, measurable increase in crack length will depend upon the resolution of the camera. For example, when using a high resolution camera with a spatial resolution of 20 μm, an increase in the crack length would need to be greater than 20 μm to be visible and, thus considered crack propagation. According to embodiments of the present invention, the hydrogels are made tough to resist crack propagation under a single cycle of mechanical load. The principle for toughening various types of hydrogels is to implement dissipation mechanisms such as fracture of short chains, reversible crosslinking and pullout of fibers into stretchy polymer networks. However, the toughening mechanisms do not contribute to anti-fatigue properties of existing tough hydrogels under cyclic loads, because the energy to resist fatigue-crack propagation in such tough hydrogels is the energy required to rupture a layer of polymer chains (i.e., the intrinsic fracture energy of hydrogels), unaffected by the additional dissipation mechanisms. The reported fatigue thresholds (i.e., the minimal fracture energy at which crack propagation occurs under cyclic loads) for synthetic hydrogels are on the order of 1-100 J/m². The controlled introduction of crystallinity in hydrogels can significantly enhance their anti-fatigue-fracture properties. The fatigue threshold of semi-crystalline hydrogel can exceed 1,000 J/m² (FIG. 6, described in more detail below).

According to embodiments of the invention, various combinations of synthetic and natural polymer may be used in forming the hydrogel membranes.

Materials suitable for use as the polymer network that maintains high elasticity and fatigue fracture resistance may be selected from polyvinyl alcohol (PVA), polyacrylamide (PAAm), polyethylene glycol (PEG), polyethylene glycol derivatives, poly-N,N-dimethylacrylamide (DMMA), cellulose, collagen, gelatin, agar, agarose, dextran, alginate, hyaluronan, chitosan, nanocomposites, and combinations thereof. The polyethylene glycol derivatives may be selected from acrylated PEG, methacrylated PEG, PEG norbornene, PEG diacrylate, PEG dimethacrylate, and combinations thereof. In those anti-fatigue polymer networks, the energy per unit area required to fracture the unit area of materials (such as nanofibrils, nanocrystalline, nanophases) for fatigue-crack propagation is much higher than the energy to fracture the corresponding amorphous polymer chains, leading to extremely high fatigue thresholds of the hydrogels.

The superabsorbent polymers that are encapsulated in the hydrogel membranes may be selected from any charged polymers. Exemplary superabsorbent charged polymer materials may include, but are not limited to, polyacrylic acid (PAA), poly(2-acrylamido-2-methyl-1-propanesulfonic acid) (PAMPS), poly [2-(methacryloyloxy)ethyl] trimethyl ammonium chloride), polyethylenimine, poly(vinylbenzyl)trimethylammonium chloride, poly(3-acrylamidopropyl)trimethylammonium chloride, polydimethylaminoethyl methacrylate, and combinations thereof. Preferably, the superabsorbent polymers may be in particle form. However, other shapes and forms may be used such as, but not limited to, granules, powders, pellets, cylinders, flakes, beads, rings, and irregular shapes. Further, combinations of one or more of these shapes could also be used. It is noted that the particular shape(s) of the superabsorbent polymers may affect the swelling speed and, thus, the shape/combination of shapes may be chosen to tailor the swelling speed for a given application. The sizes of the superabsorbent polymer particles, granules, powders, etc., can vary depending upon factors such as the size of the assembled hydrogel structure, the target insertion site, and the desired end size and/or shape, but generally range from about 100 μm to 2 mm in diameter (or largest overall dimension for shapes other than circular). More preferably, the superabsorbent polymer particles, granules, powders, etc. are generally less than about 1.5 mm, more preferably less than about 1 mm, more preferably less than about 900 μm, more preferably less than about 850 μm, more preferably less than about 800 m, more preferably less than about 700 μm, more preferably less than about 650 μm, more preferably less than about 600 μm, more preferably less than about 550 μm, more preferably less than about 500 μm, and even more preferably less than about 450 μm in diameter (or largest overall dimension for shapes other than circular).

According to an exemplary embodiment, the superabsorbent polymer particles, granules, powders, etc., may range in size from about 200 μm to about 400 μm in diameter.

According to embodiments of the invention, after the anti-fatigue hydrogel membrane is formed, macropores (generally understood as pores >50 μm, or >75 μm diameter) are formed in the membrane. Such macropores may be formed using conventional techniques, and in an exemplary embodiment, macropores are formed by laser cutting, which provides a large scale and high resolution technique. Other methods, such as punching pores by a needle array after the membrane is formed, can also be suitably used. In addition, gas-forming methods as well as freeze-defrost methods (which are known methods) may be used to fabricate macroporous hydrogels. The macropores are preferably no greater than about 500 μm, and preferably range from about 100 μm to about 400 μm in diameter. According to an exemplary embodiment, macropores having a diameter of about 200 μm are formed in the hydrogel membrane via laser cutting.

As noted above, according to preferred embodiments, the superabsorbent polymer particles, granules, powders, etc. range in size from about 200 μm to about 400 μm in diameter, while the macropores are preferably sized to be no greater than 500 μm. The superabsorbent polymer material is in a solid state and, thus, the above-described compression to form the assembled hydrogel structure results in the superabsorbent polymer material aggregating so that it remains within the assembled hydrogel structure rather than exiting through the macropores. In some embodiments, the superabsorbent polymer particles, granules, powders, etc. may be sized larger than the macropores. In any case, upon ingestion, water enters the assembled hydrogel structure through the macropores, causing the superabsorbent to absorb the water and expand.

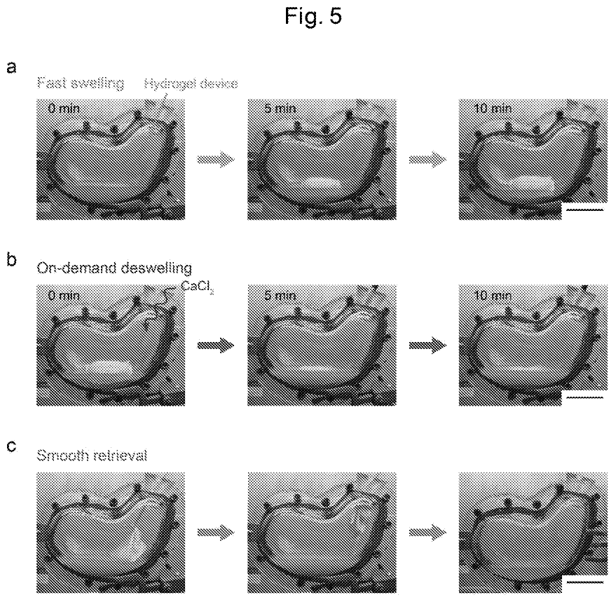

According to an embodiment of the present invention, when the assembled hydrogel structure is dropped in water or SGF, the crumpled or compressed structure (e.g., pill, capsule, etc.) restores its initial uncompressed/uncrumpled form immediately, particularly within about 5 seconds. This restoration to the initial uncompressed/uncrumpled form may include, for example, a smoothening out/straightening of the crumpled housing surfaces. In particular, the macropores provide communication between the exterior of the assembled hydrogel structure and the interior of the assembled hydrogel structure—and, thus, communication between an environment outside of the assembled hydrogel structure and the superabsorbent polymer disposed within the assembled hydrogel structure. As such, when the assembled hydrogel structure is placed in water or SGF, the water or SGF (or portions of SGF sized small enough to pass through the macropores) infiltrates through the macropores in the hydrogel membrane, and the superabsorbent polymer in the capsule then swells by absorbing and retaining target molecules (i.e., water molecules). It is noted that while additional chemical components present in the gastric fluid (e.g., Na+, Cl−, H₂O, H+) are smaller than the macropores in the hydrogel membrane and, thus, capable of passing through the macropores and interacting with the superabsorbent, only the water molecules drive the swelling of the superabsorbent. It is to be understood, however, that it is possible and within the scope of embodiments of the present invention to utilize a different type of superabsorbent which is triggered by a substance other than water. As such, other suitable superabsorbents and triggers may be readily determined by one skilled in the art depending upon the use of the expandable hydrogel structure and the environment in which the expandable hydrogel structure is utilized. The hydrogel structure responds by expanding in volume to accommodate the superabsorbent swelling (such expansion is fast and with a large ratio). The expanded hydrogel structure may be retained at the target site for a prolonged period of time. In particular, if the stomach is the target site, then the assembled hydrogel structure (e.g., pill, capsule, etc.) can be ingested such that when it reaches the stomach, it is triggered to expand to a size and shape that holds it within the stomach (i.e. prevents it from being passed through the pylorus) for a desired amount of time (FIGS. 5a-c). FIGS. 5a-c depict the ex vivo performance of the expandable hydrogel devices in a plastic stomach model containing porcine gastric fluid, including time-lapse images of swelling (FIG. 5a), calcium chloride triggered deswelling (FIG. 5b), and retrieval of the expanded hydrogel devices (FIG. 5c).

According to embodiments of the present invention, the hydrogel structure is provided with a mechanical robustness that enables the expanded hydrogel structure to withstand the peristalsis and mechanical contractility of the stomach for extended periods of time (i.e., at least 3 weeks, more preferably at least 4 weeks). In addition, by adopting compliant hydrogel materials in the present structures, embodiments of the present invention provide hydrogel structures having a mechanical modulus that matches soft living tissue to help reduce the foreign body sensation of the expanded hydrogel within the stomach. In particular, the modulus of an embodiment of the present invention of hydrogel structure is on the order of 10 kPa, which is on the same order for soft living tissues. As such, it will not induce any damage on the stomach mucosal surface or result in other side effects caused by conventional stiffer materials.

FIGS. 6a-d depict the comparison of long-term strength under cyclic tensile test between two hydrogel membranes, where FIG. 6a illustrates the undeformed state (left) and the deformed state (right) of the porous antifatigue PVA hydrogel membrane. The sample remains stable after 1, 1000, and 10,000 cycles of tensile loading, FIG. 6b illustrates the PVA hydrogel membrane can sustain true stress of at least 1.2 MPa for 10,000 cycles of tensile loading, FIG. 6c illustrates the polyacrylamide-agar hydrogel can reach the maximum true stress of 1.2 MPa but ruptures within 110 cycles of tensile loading, FIG. 6d illustrates the PVA hydrogel membrane can sustain true stress of 4.3 MPa for 9,000 cycles of tensile loading.

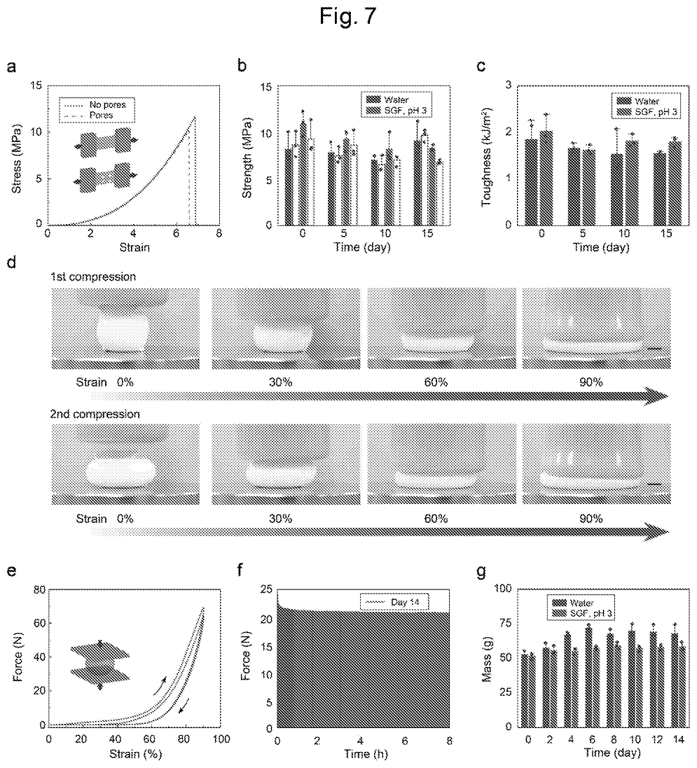

FIGS. 7a-g illustrate the mechanical robustness of the expandable hydrogel device, where FIG. 7a illustrates the true stress-stretch curves of the polyvinyl alcohol hydrogel membranes with and without pores, which have been immersed in SGF at 37° C. for 12 h, FIG. 7b illustrates the tensile strength of the hydrogel membranes with (open) and without (filled) pores, which have been immersed in water or SGF at 37° C. for 0-15 days, FIG. 7c illustrates the fracture toughness of the hydrogel membranes, which have been immersed in water or SGF at 37° C. for 0-15 days, FIG. 7d illustrates the time-lapse images of an SGF-saturated expandable hydrogel device exposed to a maximum compressive force of 70 N and a strain of 90%, FIG. 7e illustrates the force-strain curves of the SGF-saturated expandable hydrogel device exposed to a maximum compressive force of 70 N and a strain of 90% for two cycles, FIG. 7f illustrates the measured compressive forces applied to an expandable hydrogel device on day 14 (the expandable hydrogel device is immersed in SGF, and sustain 1,920 cycles of 40% compressive strains for 8 h per day), and FIG. 7g illustrates the measured mass of the expandable hydrogel device after 1,920 cycles of 40% compressive strain for 8 h per day over 14 days.

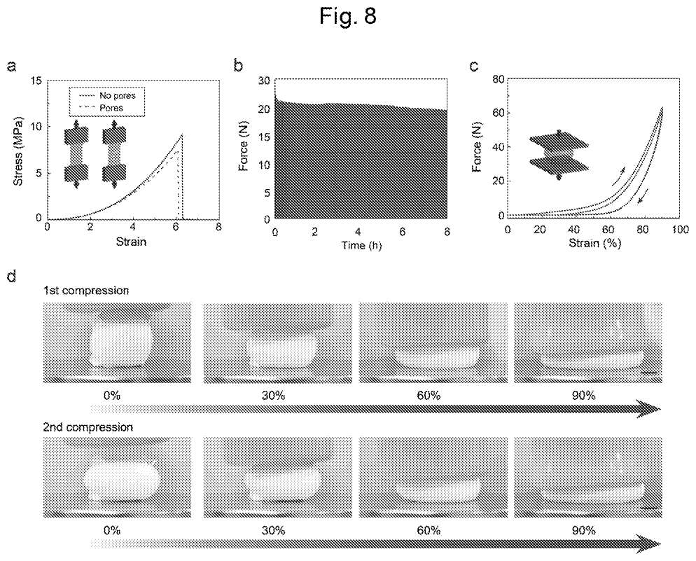

FIGS. 8a-d illustrate the mechanical robustness of the expandable hydrogel device in water, where FIG. 8a illustrates the true stress-stretch curves of the PVA hydrogel membranes with and without pores, which have been pre-immersed in water at 37° C. for 12 h, FIG. 8b illustrates the measured compressive forces applied to an expandable hydrogel device, which is kept in water under 1,920 cycles of compressive strain for 8 h on day 14, FIG. 8c illustrates the force-strain curves of a water-saturated expandable hydrogel device exposed to a maximum compressive force of 63 N and a strain of 90% for two cycles, and FIG. 8d illustrates the time-lapse images of the water-saturated expandable hydrogel device exposed to a maximum compressive force of 63 N and a strain of 90%.

To ensure the long-term robustness of the hydrogel structure in the gastric environment, its mechanical performance is evaluated, including the membrane material and the overall structure. The stomach generates hydrodynamic flows and cyclic compressive forces in order to grind food into smaller particles, mix them with gastric fluids, and empty them through the pylorus. We found that anti-fatigue hydrogels can be used as the anti-fatigue membranes for the hydrogel structure under cyclic mechanical loads (FIG. 6). The freeze-thawing treatment introduced nano-crystalline domains into the hydrogel membrane, making it strong, tough, and fatigue resistant while maintaining a low modulus (FIG. 6). After being immersed in SGF at body temperature (37° C.) for over two weeks, the hydrogel membrane demonstrated high strength of over 7 MPa and high toughness of over 1,000 J m-2 (FIG. 7). The hydrogel membrane is also capable of sustaining 9,000 cycles of 4.3 MPa tensile stress, and thus maintain the robustness of the swollen hydrogel structure under repeated loads (FIG. 6).

Furthermore, the high robustness of the swollen hydrogel structure under mechanical loads is validated. The hydrogel structure could sustain large compressive strain up to 90% and high force up to 70 N (FIG. 7 and FIG. 8). Considering the dimension of the hydrogel structure, the effective compressive stress was calculated as ˜70 kPa, which is much higher than the maximum gastric pressure (i.e., ˜10 kPa). In addition, 1,920 cycles of 40% compressive strain is applied on a hydrogel structure in SGF for 8 h every day (i.e., 26,880 cycles in total for 14 days). The steady-state compressive force reached 20 N over 14 days, corresponding to an effective compressive stress of ˜10 kPa (FIG. 7). We also recorded the mass of the swollen device after cyclic compression every day, and no mass loss was detected over two weeks (FIG. 7). In contrast, an alternative hydrogel structure made of a tough hydrogel membrane but with short fatigue life showed severe softening and loss of mass after 1,920 cycles of 40% compressive strain on the first day.

The hydrogel structures of the embodiments of the present invention, once positioned at a target site and expanded, may remain at the target site until it is triggered by a suitable material which causes the expanded hydrogel structure to deflate or otherwise shrink.

For example, removal of the expanded hydrogel structure from a target site may be facilitated using a suitable deflation trigger which causes the near-instantaneous deflation or shrinkage of the hydrogel structure (i.e., completes the desired deflation within about 10 min, e.g., as demonstrated in FIG. 4). Suitable deflation triggers may be determined, for example, by the type of superabsorbent material contained within the hydrogel structure, the type of materials forming the hydrogel membrane, and the macropore size of the hydrogel membrane. Some suitable deflation triggers include, but are not limited to, Fe³⁺, Ca²⁺, and glutathione for polyacrylic acid superabsorbent polymers.

According to an exemplary embodiment, concentrated CaCl₂) (preferably in the form of a concentrated CaCl₂) solution) was used as the deflation trigger, which is further illustrated in FIGS. 4a-d. In particular, FIG. 4a illustrates the time-lapse images of the CaCl₂) (0.6 M) induced deswelling of the expanded hydrogel device in water (pH 7), FIG. 4b illustrates the time-lapse images of the CaCl₂) (0.6 M) induced deswelling of the expanded hydrogel device in SGF, FIG. 4c illustrates volumetric deflation kinetics of the expanded hydrogel device induced by CaCl₂) (0.6 M) in different media, and FIG. 4d illustrates volumetric deflation kinetics of the expanded hydrogel device induced by CaCl₂) (0.03 M) in different media.

The swollen superabsorbent polymers de-swell owing to the interaction between the superabsorbent material and deflation trigger. In particular, in the case of a superabsorbent containing carboxyl groups (such as polyacrylic acid), CaCl₂) may be used as a deflation trigger. In this case, the calcium ions from CaCl₂) effuse through the hydrogel membrane macropores and react with the superabsorbent's carboxyl groups resulting in the swollen superabsorbent particles shrinking into white powders. This shrinkage of the superabsorbent particles results in deflation of the expanded hydrogel structure. Typically, most of the shrunken superabsorbent particles remain within the hydrogel structure, although some may possibly escape through the macropores in the hydrogel membrane. As such, the superabsorbent is preferably biocompatible. The remaining shrunken hydrogel structure is highly flexible and deformable, and can thus pass through the GI tract and exit the body without causing any obstruction.

According to various embodiments, the deflated hydrogel structure may readily exit the stomach through pylorus. Such deflation occurs quickly (preferably within about 30 minutes, more preferably within about 25 minutes, more preferably within about 20 min, and even more preferably within about 15 minutes). According to a preferred embodiment, the deflation occurs within about 10-18 minutes of exposure to a suitable deflation trigger (e.g., as shown and described below in connection with FIG. 4). Such deflation is considered to occur upon at least about an 80% weight loss of the expanded hydrogel structure, more preferably within at least about an 85% weight loss, more preferably within at least about a 90% weight loss of the expanded hydrogel structure. For example, as shown in FIG. 4, in an exemplary embodiment, the maximum weight loss of the expanded hydrogel structure was about 96%, with a minimum weight decrease being about 80%. It is noted that the density of the hydrogel before and after deflation is around 1, and, thus, this decrease can also be considered as volume loss.

The expandable hydrogel structures of the embodiments of the present invention provide a combination of desirable properties which make it particularly suitable for insertion in the stomach: (i) the hydrogel structure can be made small enough for oral administration, (ii) the expansion of the hydrogel structure is fast and large enough for the expanded structure to retain itself for a long period of time in the stomach without its evacuation through the pylorus, (iii) the robustness of expanded hydrogel structure ensures its integrity and functionality in a peristaltic and contractile stomach, (iv) the on-demand deflation allows the smooth and safe excretion out of the human body, and (v) the whole process is biocompatible and non-invasive with no need of surgery or sedation. The above unique characteristics of the embodiments of the present invention of expandable hydrogel structures enables a set of biomedical functions and applications, including bariatric intervention for nutritional modulation for the GI tract (e.g., to control obesity), ingestible electronics for more accurate and intimate recording of body signals (e.g., body temperature, gastric juice pH, and glucose level), and extended drug-release systems that release their therapeutic payload on daily basis to overcome the non-adherence problem. In such drug-release systems, the expandable hydrogel structure further includes one or more therapeutic agents disposed within the cavity, where such therapeutic agents are released from the expanded hydrogel structure (e.g., by diffusion through the hydrogel membrane, by passage through the macropores, and other known slow-release/controlled-release methods). In addition to use as an intragastric retentive device, the present hydrogel structures can also suitably be used in hemorrhage control.

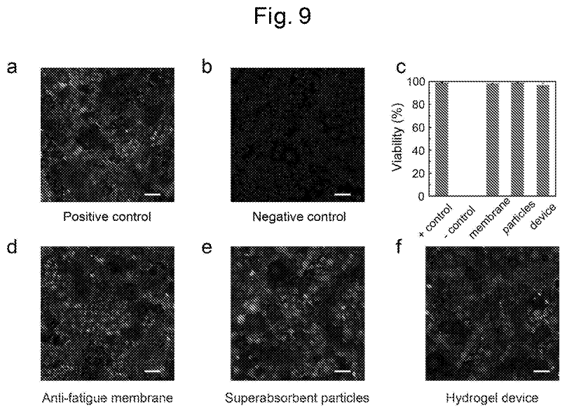

According to embodiments of the present invention, the hydrogel structure is biocompatible which should typically not cause any cell death when co-culture with Caco-2 cells (FIG. 9). In particular, FIGS. 9a-f illustrate the cell compatibility of the expandable hydrogel devices, where FIG. 9a illustrates cell viability is tested on Caco-2 cells with medium that was pre-exposed to the expandable hydrogel device and its integral parts for one day at 37° C. Cells without treatment and cells treated with 70% ethanol serve as positive (FIG. 9a) and negative (FIG. 9b) control, respectively. Cell viability after 72 h incubation with medium pre-exposed to the anti-fatigue membrane (FIG. 9d), superabsorbent particles (FIG. 9e), and the whole expandable hydrogel device (FIG. 9f) are presented, respectively. Bright large regions indicate viable cells. FIG. 9c illustrates the viability is calculated by the ratio of viable cells to all cells in the images.

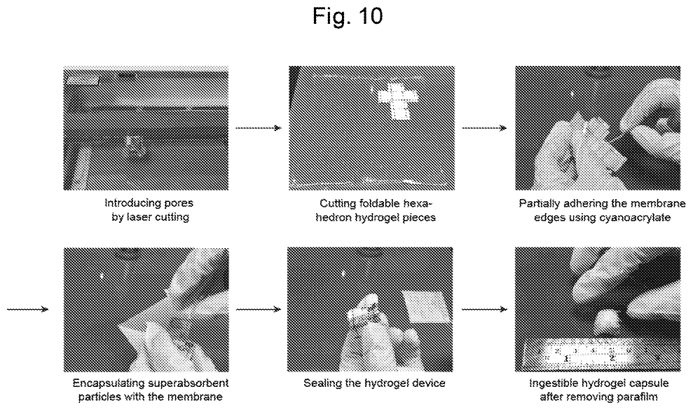

According to various embodiments, the porous anti-fatigue hydrogel membrane is formed into the desired shape by cutting it along a template. For example, the hydrogel membrane may be provided in a non-stick support on which the template may be depicted. After cutting the hydrogel membrane to the template, the resultant structure is folded or otherwise shaped into the desired end shape (e.g., as illustrated in FIG. 10, the superabsorbent particles and a porous hydrogel membrane are assembled into an expandable hydrogel device with cube shape). According to embodiments of the invention, the hydrogel membrane forms a housing with a cavity provided therein. The cavity is loaded with a predetermined amount of superabsorbent polymer, and the housing is sealed thereby sealing the superabsorbent polymer within the housing. According to an exemplary embodiment, the housing is sealed using any conventional methods for sealing hydrogels, such as by adhering edges of the housing together via superglue (e.g., LOCTITE® super glue) or other conventional adhesive materials. If a support was provided, then it is removed, and the hydrogel shape is crumpled or otherwise compressed into a smaller structure (e.g., pill or capsule) that can be ingested, implanted, or otherwise inserted into a target site. This compressed structure may be referred to as the assembled hydrogel structure.

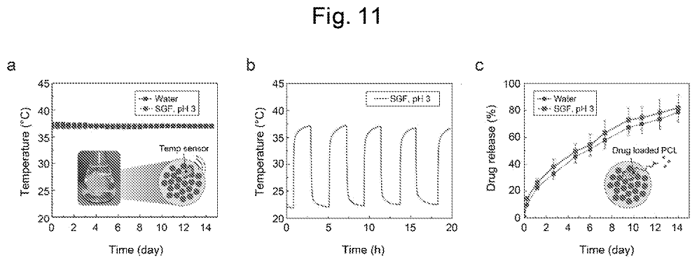

FIGS. 11a-c illustrate in vitro sensing and drug release capacity of the expandable hydrogel devices, where FIG. 11a illustrates the temperature pattern of media placed in an incubator set to 37° C. and monitored by use of an expandable hydrogel device comprising a temperature sensor, FIG. 11b illustrates the temperature pattern with alternating placement of the expandable hydrogel device at room temperature, and FIG. 11c illustrates the extended caffeine release from a polycaprolyactone-based formulation incorporated in the expandable hydrogel device at 37° C.

A temperature sensor (DST nanoRF-T, Star-Oddi™) is embedded in the hydrogel structure. The recorded temperature of the hydrogel structure under different conditions for 29 days (FIG. 11) reveals the capability to monitor in-situ physiological signals for an extended period of time. FIG. 11 shows a plot of the temperature profiles, which is enabled by the temperature sensor embedded inside the hydrogel structure.

A PCL drug depot containing caffeine is incorporated into the hydrogel structure. Caffeine release from the hydrogel structure was monitored (FIG. 11). The drug depot stays inside the hydrogel structure, but is able to release the drug over a long period of time.

The expandable hydrogel structures and methods of the present invention will be further illustrated with reference to the following examples which are intended to aid in the understanding of the embodiments of the present invention, but which are not to be construed as a limitation thereof.

### EXAMPLES

### Synthesis of the Hydrogel Membranes

All types of anti-fatigue hydrogel membranes were prepared from polyvinyl alcohol powders (PVA; Mw 146,000-186,000, 99+% hydrolyzed, Sigma-Aldrich). An aqueous solution of 10 wt % PVA was dissolved by stirring at 75° C. for 6 h, and mixed and defoamed by using a centrifugal mixer (AR-100, Thinky™) for 1 min. The solution was cast in a 0.6-mm-thick custom-made glass mold, frozen at −20° C. for 8 h, and thawed at 25° C. for 3 h. The 8-h-freezing and 3-h-thawing was defined as one freeze-thaw cycle. Samples that underwent one freeze-thaw cycle were the soft hydrogel membranes (Young's modulus 2.6 kPa), and samples that underwent four freeze-thaw cycles were the medium hydrogel membrane (Young's modulus 47 kPa). The PVA hydrogels, after four freeze-thaw cycles, were further air-dried at 37° C. for 1 h, and annealed at 100° C. for 1 h, so that we obtained the stiff hydrogel membranes (Young's modulus 1.13 MPa). For radiographic visualization in vivo, 23 wt % of radio-opaque barium sulfate (Sigma-Aldrich™) was incorporated in the hydrogel membrane. The addition of barium sulfate did not affect the mechanical properties as suggested by identical stress-strain curves and moduli (˜46 kPa for both). All types of resulting PVA hydrogels were left in the molds for 2 days at room temperature before further use.

An alternative hydrogel membrane was prepared using a polyacrylamide-agar tough hydrogel. Agar (2 g; Sigma-Aldrich™), acrylamide (18 g; Sigma-Aldrich™), Irgacure™ 2959 (0.284 g; Sigma-Aldrich) as the photoinitiator, and N,N′-methylenebisacrylamide (0.007 g; Sigma-Aldrich) as the crosslinker were dissolved in 75 mL water at 90° C. The solution was degassed thoroughly and poured in the custom-made glass mold (0.6 mm thick). The precursor solution was allowed to cool in the mold at room temperature to form the agar network, and then exposed to UV irradiation (365 nm wavelength, 8 W) for 1 h to form the polyacrylamide network. Unreacted monomer and photoinitiators are leached out from the membrane for 2 days by water.

### Fabrication of the Expandable Hydrogel Devices

For water permeation into the expandable hydrogel device, the hydrogel membranes were introduced with uniform pores (˜200 μm in diameter, two pores per cm²) using laser cutting (Epilog™). The hydrogel membranes were then trimmed based on a Parafilm® template (Bemis™) and assembled into a pocket or cube structure for subsequent loading with a specific amount of superabsorbent particles (sodium polyacrylate homopolymers; Waste Lock 770, M2 Polymer Tech™). The edges of the assembled pocket or cube structure were adhered using biocompatible ethyl cyanoacrylate glue (LOCTITE®). After the Parafilm template was removed, the expandable hydrogel device was further crumpled into a pill size.

### Swelling Tests

The prepared expandable hydrogel devices were submerged in aqueous media for swelling tests, which included water, simulated gastric fluid (SGF, pH 3), or porcine gastric fluid. Compendial SGF was prepared with sodium chloride (150 mM) and hydrochloric acid (1 mM) in water. Porcine gastric fluid was withdrawn endoscopically when we performed another observational endoscopic study in the pig, and stored at −80° C. During swelling studies, the increase of volume over time was monitored using a DSLR camera (NIKON® D7000), and the mass of expandable hydrogel devices was monitored using analytical balance (Denver Instrument™). Swelling of superabsorbent particles was recorded using microscopy (Nikon Eclipse™ LV100ND). The volume at each time point was obtained by the area to the 1.5th power, where the area was accessible from the time-lapse images. The volume change was expressed as V/V₀, normalized by the initial volume V₀.

Swelling of Bulk and Porous Hydrogels

The air-dried bulk hydrogel and the freeze-dried porous hydrogel were prepared from polyacrylamide-agar following the same procedure described for synthesis of the hydrogel membranes (see above) with modifications. Those modifications included that the as-prepared hydrogels were dried at room temperature for 2 days to form bulk hydrogels. To prepare porous hydrogels, the hydrogels were fully swollen in water for 2 days, frozen at −20° C. for 1 day, and lyophilized for 3 days. The mass of each hydrogel was recorded over time during their swelling. The measured swelling times of the air-dried and freeze-dried hydrogels were normalized to the initial sample size of 5 mm.

### Deswelling Test

Deswelling characteristics of the expanded hydrogel devices were evaluated by inducing deswelling with 0.6 M or 0.03 M calcium chloride in swelling media of water or SGF. The mass and volume of the expandable hydrogel devices were recorded over time in the deswelling tests following the procedures described in the swelling tests.

### Mechanical Testing of the Hydrogel Membranes

To assess the mechanical properties of hydrogel membranes under simulated physiological conditions, hydrogel membranes with or without pores were incubated in various media of water (pH 7) and SGF at 37° C. before performing mechanical testing. They were cut into dog-bone specimen with 6.5 mm in width, 15 mm in gauge length, and 0.75 mm in thickness for all samples, and 1.2 mm in crack length for notched samples. At 12 h, 5 days, 10 days, and 15 days after incubation, true stress-strain curves of the unnotched hydrogel membranes were measured using a mechanical testing device (Z2.5, Zwick-Roell™) with a 20 N load cell. The strain rate was imposed to 2 s⁻¹.

To test the fatigue properties of hydrogel membranes, we performed cyclic tensile loading on the hydrogel membranes with pores in a water bath (pH 7) at 25° C. with a benchtop mechanical tester (UStretch™, CellScale™), using a 44 N load cell. Forces applied were recorded over time. The strain rate was imposed to 5 s⁻¹.

### Mechanical Testing of the Expandable Hydrogel Devices

The expandable hydrogel devices, which were fully swollen in SGF or water for 1 h beforehand, were exposed to cyclic compression using a mechanical testing device (Z2.5, Zwick-Roell™) with a 2,500 N load cell and a cylindrical soft indenter (Ecoflex™, diameter 70 mm). The strain rate imposed was 2 s⁻¹. The maximum engineering strain was 40% for long-time compression, and the expandable hydrogel devices (diameter ˜4.8 cm, cross-section area ˜20 cm² at undeformed state) in the medium underwent 8-h cyclic loading every day. In the short-run test, the maximum engineering strain was 90%, and the devices (diameter ˜3.6 cm, cross-section area ˜10 cm² at undeformed state) in the air underwent two cycles. The effective compressive stress was calculated by dividing the compressive force by the cross-section area of the undeformed expandable hydrogel device. Hertz model was used to obtain the effective modulus of whole expandable hydrogel devices from the compression curves.

### Cytotoxicity Analysis

Cell viability was tested on Caco-2 cells (American Type Culture Collection). Caco-2 cells (clone: C2BBe1, passage 48-58) were cultured in Dulbecco's modified eagle medium (Life Technologies) supplemented with 10% fetal bovine serum (Sigma-Aldrich™), 1× non-essential amino acids solution (Life Technologies), 1× GlutaMAX™ (Life Technologies), and penicillin/streptomycin (Life Technologies). 5 mL of fresh culture medium were introduced to the expandable hydrogel device and its components, including the hydrogel membrane and superabsorbent particles, for one day at 37° C. (hereafter referred to as pre-exposed medium). One day after the Caco-2 cells were seeded, the culture medium was replaced with the pre-exposed medium, and the cells were co-incubated with the pre-exposed medium for 72 h without changing the medium. Cells treated with 70% ethanol and untreated cells were used as a negative control and positive control, respectively. Finally, cell viability was analyzed using a commercial assay according to the manufacturer's protocol (LIVE/DEAD® viability/cytotoxicity kit for mammalian cells, Life Technologies). The cells were imaged using a Leica™ SP8 upright confocal microscope.

### In Vitro Temperature Sensing of the Expandable Hydrogel Devices

The temperature sensor (DST nanoRF-T, Star-Oddi™), 1.5 cm in length and 0.5 mm in diameter was embedded in the expandable hydrogel device, which was then allowed to swell in water or SGF. The entire expandable hydrogel device in media was placed in an incubator set to 37° C., or alternatively placed at 37° C. and at room temperature. The antenna was mounted on the wall of the incubator, enabling real-time temperature reading in the expandable hydrogel device.

### In Vitro Drug Release of the Expandable Hydrogel Devices

Formulations containing 2.5 mg caffeine (Sigma-Aldrich™), 0.14 g pluronic P407 (Sigma-Aldrich™), and 1 g linear polycaprolactone (PCL; Mw 45,000, Sigma-Aldrich™) were combined, melted at 90° C., and mixed vigorously. The molten mixture was transferred into a small acrylic mold 1.5 cm in length and 0.5 mm in diameter, heated at 90° C. for 2 h, and air-cooled to room temperature. The PCL drug depot was then incorporated into the expandable hydrogel devices. Caffeine release from the expandable hydrogel device was monitored using UV-Vis spectrometry at 275 nm (BioMate 3S, Thermo-Fisher™).

### Ex Vivo Study in a Stomach Model

A stomach model custom-made of plastics and containing 75 mL porcine gastric fluid was used as an accessible ex vivo model. To trigger the deswelling of the fully expanded hydrogel device, 7.5 mL calcium chloride solution (50 wt %) was added 30 min after the insertion and swelling of the hydrogel device in gastric fluid. A snare catheter (Captivator™ II Single-Use Snare, Boston Scientific™) was used to retrieve the shrunken hydrogel device through the opening. The process was monitored using a camera (EOS 70D, Canon™).

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

1. An expandable hydrogel structure comprising:
a housing fabricated of a hydrogel membrane, the housing having a cavity therein;
a superabsorbent material disposed within the housing cavity, the superabsorbent material having an initial superabsorbent size; and
a plurality of macropores in the hydrogel membrane,
wherein the housing has an initial housing size, and wherein the housing has an expanded housing size upon exposure of the superabsorbent material to an expansion trigger, the expanded housing size being at least about 50 times to at least about 100 times the initial housing size.

2. The expandable hydrogel structure of claim 1, wherein the superabsorbent material is sealed within the housing, and wherein the plurality of macropores provides communication between an exterior of the housing and the superabsorbent material.

3. The expandable hydrogel structure of claim 2, wherein the superabsorbent material has an initial superabsorbent size greater than a size of the plurality of macropores, and wherein the macropores are sized for passage of the expansion trigger through one or more of the plurality of macropores.

4. The expandable hydrogel structure of claim 3, wherein the expansion trigger is absorbable by the superabsorbent material, and wherein absorption of the expansion trigger expands the superabsorbent material from an initial superabsorbent size to an expanded superabsorbent size.

5. The expandable hydrogel structure of claim 4, wherein the expanded superabsorbent size stretches the hydrogel membrane to the expanded housing size.

6. The expandable hydrogel structure of claim 1, wherein the hydrogel membrane is fabricated of an anti-fatigue polymer network infiltrated with water, which resists a crack propagation under cyclic loading.

7. The expandable hydrogel structure of claim 6, wherein the antifatigue polymer network is selected from polyvinyl alcohol (PVA), polyacrylamide (PAAm), polyethylene glycol (PEG), one or more polyethylene glycol derivatives, poly-N,N-dimethylacrylamide (DMMA), cellulose, collagen, gelatin, agar, agarose, dextran, alginate, hyaluronan, chitosan, and combinations thereof.

8. The expandable hydrogel structure of claim 7, wherein the one or more polyethylene glycol derivatives are selected from acrylated PEG, methacrylated PEG, PEG norbornene, PEG diacrylate, PEG dimethacrylate, and combinations thereof.

9. The expandable hydrogel structure of claim 7, wherein the antifatigue polymer network includes one or more nanostructures.

10. The expandable hydrogel structure of claim 1, wherein the superabsorbent material is selected from charged polymer networks.

11. The expandable hydrogel structure of claim 1, wherein the superabsorbent material is selected from polyacrylic acid (PAA), poly(2-acrylamido-2-methyl-1-propanesulfonic acid) (PAMPS), poly [2-(methacryloyloxy)ethyl] trimethyl ammonium chloride), polyethylenimine, poly(vinylbenzyl)trimethylammonium chloride, poly(3-acrylamidopropyl)trimethylammonium chloride, polydimethylaminoethyl methacrylate, and combinations thereof.

12. The expandable hydrogel structure of claim 1, wherein the expansion trigger is one or more of water and gastric fluid.

13. The expandable hydrogel structure of claim 1, wherein the expandable hydrogel structure is an ingestible pill.

14. The expandable hydrogel structure of claim 1 further comprising one or more therapeutic agents disposed within the cavity.

15. A weight-loss balloon for placement in a patient's stomach comprising:
a housing fabricated of a hydrogel membrane, the housing having a cavity therein;
a superabsorbent material disposed and sealed within the housing cavity, the superabsorbent material having an initial superabsorbent size; and
a plurality of macropores in the hydrogel membrane providing fluid communication between the superabsorbent material within the cavity and an exterior of the housing,
wherein the housing has an initial housing size, and wherein the housing has an expanded housing size upon exposure of the superabsorbent material to an expansion trigger, the expanded housing size being at least about 50 times to at least about 100 times the initial housing size.

16. A method for placing a weight-loss balloon within a patient's stomach comprising:
administering to a patient an ingestible pill comprising:
a housing fabricated of a hydrogel membrane, the housing having a cavity therein;
a superabsorbent material disposed and sealed within the housing cavity, the superabsorbent material having an initial superabsorbent size; and
a plurality of macropores in the hydrogel membrane providing fluid communication between the superabsorbent material within the cavity and an exterior of the housing;
allowing gastric juice to pass through the plurality of macropores and allowing the superabsorbent material to absorb one or more expansion triggers in the gastric juice to thereby form a swollen superabsorbent material; and
allowing the swollen superabsorbent material to stretch the hydrogel membrane and form a weight-loss balloon structure;
wherein the weight-loss balloon structure is at least about 50 times to at least about 100 times larger than the ingestible pill.

17. The method of claim 16, further comprising removing the weight-loss balloon structure from the patient's stomach by administering a deflation trigger to the patient's stomach, allowing the deflation trigger to pass though the plurality of macropores and react with the swollen superabsorbent material to thereby de-swell the swollen superabsorbent material.

18. The method of claim 17, wherein the deflation trigger comprises one or more high valent ions.

19. The method of claim 17, wherein the deflation trigger comprises one or more calcium ions.

20. A method for placing an expandable hydrogel structure within a patient's stomach comprising:
administering to a patient an ingestible pill comprising:
a housing fabricated of a hydrogel membrane, the housing having a cavity therein;
a superabsorbent material disposed and sealed within the housing cavity, the superabsorbent material having an initial superabsorbent size; and
a plurality of macropores in the hydrogel membrane providing fluid communication between the superabsorbent material within the cavity and an exterior of the housing;
allowing an expansion trigger to pass through the plurality of macropores and allowing the superabsorbent material to absorb the expansion trigger to thereby form a swollen superabsorbent material; and
allowing the swollen superabsorbent material to stretch the hydrogel membrane and form an expanded hydrogel balloon structure;
wherein the expanded hydrogel balloon structure is at least about 50 times to at least about 100 times larger than the ingestible pill.

21. The expandable hydrogel structure of claim 14, wherein the one or more therapeutic agents are disposed within the cavity such that the one or more therapeutic agents are released from the expanded housing.

22. The expandable hydrogel structure of claim 21, wherein the expanded housing is configured to remain expanded for at least fourteen days.

23. The expandable hydrogel structure of claim 22, wherein the hydrogel membrane is configured to provide release of the one or more therapeutic agents from the expanded housing by diffusion through the hydrogel membrane and/or by release through the plurality of macropores in the hydrogel membrane.

24. The expandable hydrogel structure of claim 23, wherein the hydrogel membrane provides controlled release of the one or more therapeutic agents from the expanded housing.

25. The expandable hydrogel structure of claim 24, wherein the controlled release releases the one or more therapeutic agents from the expanded housing for at least fourteen days.

26. The expanded hydrogel structure of claim 25, wherein the one or more therapeutic agents is released from the expanded housing on a daily basis for at least fourteen days.

27. The expandable hydrogel structure of claim 14, wherein the expandable hydrogel structure is an extended therapeutic agent release device, and wherein the hydrogel membrane is configured to provide controlled release of the one or more therapeutic agents from the expanded housing for at least fourteen days.

28. The expandable hydrogel structure of claim 27, wherein the hydrogel membrane is configured to provide controlled release of the one or more therapeutic agents from the expanded housing on a daily basis for at least fourteen days.

29. The method of claim 20, wherein the ingestible pill further comprises one or more therapeutic agents disposed within the cavity, and wherein the method further comprises:
after allowing the swollen superabsorbent material to stretch the hydrogel membrane and form the expanded hydrogel balloon structure, allowing release of the one or more therapeutic agents from the expanded housing.

30. The method claim 29, further comprising controlling the release of the one or more therapeutic agents from the expanded housing.

31. The method of claim 30, further comprising allowing the expanded housing to remain expanded for at least fourteen days, and wherein controlling the release of the one or more therapeutic agents from the expanded housing comprises releasing the one or more therapeutic agents from the expanded housing for at least fourteen days.

32. The method of claim 31, wherein the expanded housing is allowed to remain expanded for at least three weeks.

33. The method of claim 32, wherein the expanded housing is allowed to remain expanded for at least four weeks.

34. The method of claim 30, wherein controlling the release of the one or more therapeutic agents from the expanded housing comprises releasing the one or more therapeutic agents from the expanded housing on a daily basis for at least fourteen days.

35. The method of claim 29, wherein allowing release of the one or more therapeutic agents from the expanded housing comprises allowing diffusion of the one or more therapeutic agents through the hydrogel membrane and/or allowing release of the one or more therapeutic agents through the plurality of macropores in the hydrogel membrane.

36. The method of claim 35, further comprising controlling the release of the one or more therapeutic agents from the expanded housing.

37. The method of claim 36, further comprising allowing the expanded housing to remain expanded for at least fourteen days, and wherein controlling the release of the one or more therapeutic agents from the expanded housing comprises releasing the one or more therapeutic agents from the expanded housing for at least fourteen days.

38. The method of claim 37, wherein the expanded housing is allowed to remain expanded for at least three weeks.

39. The method of claim 38, wherein the expanded housing is allowed to remain expanded for at least four weeks.

40. The method of claim 36, wherein controlling the release of the one or more therapeutic agents from the expanded housing comprises releasing the one or more therapeutic agents from the expanded housing on a daily basis for at least fourteen days.

41. A method for providing extended release of one or more therapeutic agents to a target site comprising:
placing an expandable hydrogel structure within the target site, the expandable hydrogel structure comprising:
a housing fabricated of a hydrogel membrane, the housing having a cavity therein;
a superabsorbent material disposed and sealed within the housing cavity, the superabsorbent material having an initial superabsorbent size;
a plurality of macropores in the hydrogel membrane providing fluid communication between the superabsorbent material within the cavity and an exterior of the housing; and
one or more therapeutic agents disposed within the cavity;
allowing an expansion trigger to pass through the plurality of macropores and allowing the superabsorbent material to absorb the expansion trigger to thereby form a swollen superabsorbent material;
allowing the swollen superabsorbent material to stretch the hydrogel membrane and form an expanded hydrogel balloon structure configured to remain expanded for at least fourteen days; and
allowing release of the one or more therapeutic agents from the expanded hydrogel balloon structure for at least fourteen days;
wherein the expanded hydrogel balloon structure is at least about 50 times to at least about 100 times larger than the expandable hydrogel structure.

42. The method of claim 41, wherein the expanded housing is allowed to remain expanded for at least three weeks.

43. The method of claim 41, wherein the expanded housing is allowed to remain expanded for at least four weeks.

44. The method of claim 41, wherein the one or more therapeutic agents are released from the expanded hydrogel balloon structure on a daily basis for at least fourteen days.

45. The method of claim 41, further comprising, after allowing release of the one or more therapeutic agents from the expanded hydrogel balloon structure for at least fourteen days,
exposing the expanded hydrogel balloon structure to a deflation trigger;
allowing the expanded balloon structure to shrink to a shrunken structure; and
removing the shrunken structure from the target site.

46. The method of claim 41, wherein the expandable hydrogel structure is an ingestible or implantable pill.

47. The method of claim 41, wherein the target site is a stomach of a patient.
